(19) United States
(12) Patent Application Publication
(10) Pub. No.: US 2020/0170547 A1
(43) Pub. Date: Jun. 4, 2020

(54) A HUMAN INTENTION DETECTION SYSTEM FOR MOTION ASSISTANCE

(71) Applicant: Aalborg Universitet, Aalborg Ø (DK)

(72) Inventors: Shaoping Bai, Aalborg (DK); Muhammad Raza Ul Islam, Aalborg Ø (DK)

(21) Appl. No.: 16/332,747

(22) PCT Filed: Sep. 14, 2017
(86) PCT No.: PCT/DK2017/050290

§ 371 Date: Mar. 12, 2019

(30) Foreign Application Priority Data
Sep. 14, 2016 (DK) PA 2016 00534
May 5, 2017 (DK) PA 2017 70311

(51) Int. Cl. A61B 5/11; A61B 5/00; G06N 3/08; G06N 20/10

(52) U.S. Cl. CPC A61B 5/1107; A61B 5/6824; A61B 2562/0247; G06N 3/08; G06N 20/10; A61B 5/6831

(57) ABSTRACT

A device and method for human intention detection Sensor Band (HID). In preferred embodiments, it makes use of an array of force sensing resistors (FSRs) which are embedded inside a flexible band, which is capable of reading the muscle activity for different motion type and muscle forcein a human user. In one implementation of the invention two of such bands are attached to the forearm and the upper arm. From the readings of the sensors, the patterns for motion type and muscle force are then distinguished autonomously by machine learning, a Support Vector Machine (SVM) algorithm, or a neural network. The method is advantageous e.g. the detection of dexterous motion of the arms, upon which assistive exoskeleton can be controlled for motion assistance. The invention can also be applicable to hand gestures recognition and bilateral rehabilitation, besides this the invention can be used to control lower body exoskeleton as well.

### FIELD OF THE INVENTION

The present invention relates to the field of human intention detection (HID) systems, especially for motion assistance, such as for control of exoskeleton motion assistance devices or robotic devices, controlled by a motion of a limb (arm or leg). Specifically, the invention relates to detection of the dexterous arm motion (flexion/extension) and physical effort (force).

### BACKGROUND OF THE INVENTION

Assistive eoxkskeletons are wearable electro-mechanical devices attached to bodies of users with the goal to assist them in physical movements. Intention detection plays a key role in the effective motion control of assistive exoskeletons. Attempts have been made to detect the human intention by interpreting the cognitive activity. EEG and EMG sensors are employed to read the electrical signals from brain and muscles respectively. A limitation with such technologies is the computational expense in addition to the inconvenient use. The sensors have to be sticked to skin at proper place which makes it uncomfortable and inconvenient. Such a way of sensor mounting makes a huge difference if the intention detection system is designed for assistive exoskeletons. Moreover stability and reliability of sensing is also a problem due to the moist on the skin.

There have been reports on detecting posture and gripping forces. However, no previous work has been reported related to flexion/extension of the elbow joint and force exerted by the upper arm muscles in order to carry the different loads.

### SUMMARY OF THE INVENTION

In order to solve the above mentioned problems with known human intention detection systems, the invention provides, in a first aspect, a human intention detection device arranged to detect an intended motion of a human user, and to generate an output accordingly, such as for input to an actuator device, the method comprising
a first force sensing device arranged for mounting around an upper part of a limb part, such as on an upper arm, of the human user so as to allow sensing of muscle contraction activity, wherein the first force sensing device comprises a plurality of force sensors spatially distributed to allow detection of different muscle parts of the human user's upper limb part and to generate outputs accordingly, such as comprising at least 5 spatially distributed force sensitive resistors (FSRs) arranged to generate individual outputs, and
a processor device arranged to receive said outputs from the force sensors of the first force sensing device, wherein the processor device comprises a processor arranged to execute a detection algorithm in response to said outputs from the force sensors of the first force sensing device, and wherein the processor device is arranged to output in real-time an intended motion and an intended force according to an output from the detection algorithm.

Thus, the invention proposes a new HID system which is developed with FSRs, if mounted around limbs/fingers, are able to measure radially directed muscle pressures, from which a human's action intention can be detected. In preferred embodiments, the HID device comprises sensors bands with FSRs embedded inside, aided with machine learning algorithm, in order to detect the human intention for an arm's dexterous motion i.e. motion type and muscle effort or force, with high accuracy. The invention has the advantage of compact design, comfortability and furthermore it can even be worn over thin clothes which make it more convenient and easy to use.

It is based on the insight of the inventors, that it is possible to provide a detection algorithm capable of rather precisely detect a human user's intention with respect to both motion (spatial) and force (effort) of a limb (arm or leg) based on muscle contraction activity detection with force sensors mounted on an upper limb. Especially, with 5 FSRs on the user's upper arm, both intended motion and force can be detected in a manner sufficient to control a robotic arm or an exoskeleton actuator device for motion assistive purposes.

The HID device may be applied for control of assistive exoskeleton actuator devices within a large number of applications including such as: in industry, law enforcement, military, firefighting, construction, gardening etc. Here, the exoskeleton equipment can help a person to lift a higher load and/or repeat the same movement over a longer time without fatigue. Other types of application are therapy, e.g. for training, rehabilitation, and assisting of elderly or handicapped persons in performing normal daily life activities involving motion of an arm and/or a leg.

In the following, preferred features and embodiments will be described.

The HID device may comprise a second force sensing device arranged for mounting around a lower part of the same limb, such as a forearm, as said first force sensing device, wherein the second force sensing device comprises a plurality of force sensors spatially distributed to allow detection of different muscle parts of the human user's lower part of the limb, and to generate outputs accordingly, such as comprising at least 5 spatially distributed FSRs arranged to generate individual outputs, and wherein the processor device is arranged to receive outputs from the force sensors of the second force sensing device and to output in real-time the intended motion and the intended force in response to outputs from the force sensors of the first and second force sensing devices. With such force sensing device on the lower part of the same limb (arm or leg), further information can be detected, thereby allowing an even more precise motion and force detection.

Especially, the first force sensing device is designed for mounting on the upper arm of the human user. However, it is to be understood that the first force sensing device may be designed form mounting on the thigh of the human user. Especially, the first force sensing device is designed for mounting on the upper arm of the human user and the second force sensing device is designed for mounting on the forearm of the human user.

Preferably, the HID device is arranged to discriminate between a plurality of levels of said muscle contraction activity. Thus, both detection of any activity in different muscles as well as the level or force of their contractions is preferably detected and provided as input to the detection algorithm.

In some embodiments, the force sensing device comprises a strap with the plurality of force sensors, such as 5-10 force sensors, arranged on a line, on the side of the strap arranged for facing the human user's upper limb part.

The plurality of force sensors are preferably Force Sensitive Resistor (FSR) type sensors. However, other force sensing technologies may be used as well.

The detection algorithm may implement a support machine vector (SVM) or a neural network for classifying the intended motion and the intended force.

The detection algorithm may comprise a training session where the human user performs a plurality of intended motions for generating test data, wherein the detection algorithm further comprises computing accuracy in response to the test data, and wherein the detection algorithm also comprises selecting features which can be obtained from both data fusion and original signals from FSRs for use in outputting said real-time intended motion and intended force based on the training sessions results.

In scenario involving a training session as mentioned above, a machine learning algorithm may be applied to the data obtained in the training session, thereby allowing the detection algorithm to be adapted to an individual human user and the exact position of the force sensitive resistors.

Preferably, information obtained from force reading and data fusion, is used to detect the intended motion and the intended force. Especially, information through data fusion may result from combining an output of at least two force sensors comprising at least one force sensor arranged on the upper arm and at least one force sensor arranged on the forearm.

In one group of embodiments, the first device comprises a strap or a sleave arranged for mounting around an upper arm of the human user, wherein a plurality of force sensing devices, preferably FSRs, are spatially distributed on the strap or sleave so as to allow detection of contraction at a plurality of positions along biceps brachii and/or brachialis, such as at least one position along the biceps brachii and at least one position along the brachialis, when the strap or sleave is mounted on the upper arm of the human user. Especially, at least two force sensing devices, e.g. FSRs, may be positioned on the strap or sleave so as to be located at a plurality of positions along a length direction of the biceps brachii, when the strap or sleave is mounted on the upper arm of the human user. Especially, at least one force sensing device, such as an FSR, is positioned on the strap or sleave so as to be located at the brachialis, when the strap or sleave is mounted on the upper arm of the human user. Preferably, at least 4 FSRs are spatially distributed on the strap or sleave so as to allow detection of contraction at four positions along biceps brachii and/or brachialis. Specifically, the at least 4 FSRs are spatially distributed to cover at least two different positions along a length of the biceps brachii as well as at least two different positions perpendicular to the length of the biceps brachii. Preferably, 5-8 FSRs are spatially distributed to cover at least three different positions along a length of the biceps brachii as well as at least two different positions perpendicular to the length of the biceps brachii. With this strap or sleave embodiment, the detection algorithm is preferably arranged to output one or more intended motions selected from: flexion, extension, pronation and supination, in response to outputs from the plurality of force sensing devices. Specifically, the detection algorithm may be arranged to output two or more intended motions selected from: flexion, extension, pronation and supination, in response to outputs from the plurality of force sensing devices on the upper arm only, e.g. the detection algorithm may be capable of outputting all four of the mentioned types of motions in response to the force sensors on the strap or sleave, e.g. without any further inputs from other sensors than sensors on the upper arm.

The mentioned strap or sleave with force sensors may alternatively be combined one force sensing device, such as an FSR, arranged for position on a location of the forearm of the human user, such as the at least one FSR being arranged on a separate strap or sleave arranged for mounting around the forearm of the human user. Hereby further motion types may be detected.

The first force sensing device may comprise a strap or sleave on which the plurality of force sensors are mounted at different positions. Especially, the strap or sleave is made of an elastic material, such as an elastic garment.

In a second aspect, the invention provides a method for detecting an intended human motion, the method comprising
sensing a muscle contraction activity with a plurality of force sensors arranged on an upper part of a limb, such as on an upper arm, of the human user, wherein said sensors are spatially distributed to allow detection of different muscle parts of the human user's upper part of said limb,
executing a detection algorithm on a processor in response to outputs from the force sensors, and
outputting in real-time an intended motion and an intended force according to an output from the detection algorithm.

In a third aspect, the invention provides a method for controlling an exoskeleton actuator device, comprising receiving the intended motion and the intended force rom a human intention detection device according to the first aspect, and comprising controlling the exoskeleton actuator device in response to the intended motion and the intended force. Especially, the first sensing device of the human intention detection device is mounted on an upper arm of one arm of the human user, such as comprising at least 5 FSRs, and wherein the human user has an exoskeleton actuactor device mounted on the opposite arm of the human user controlled in accordance with the intended motion and an intended force output from the human intention detection device. Especially, said exoskeleton actuator device mounted on the opposite arm of the human user has at least one elbow joint actuactor which is controlled in accordance with the intended motion and an intended force output from the human intention detection device.

In a forth aspect, the invention provides computer executable program code arranged to perform the method according to the second or third aspect, when executed on a processor. E.g. a processor of a dedicated device, or a general computer. The program code may be present on a tangible medium, e.g. a memory card or the like, or it may be present on a server for downloading via the internet. Still further, the program code may be stored on an electronic chip.

In a fifth aspect, the invention provides use of the HID device of the first aspect. Especially, use for controlling a robotic arm comprising an actuator, such as a robotic arm positioned away from the human user. In another embodiment, use for controlling an exoskeleton actuator device arranged to be worn by the human user for assisting in moving one limb of the human user, such as the exoskeleton actuator device comprising at least one elbow joint actuactor arranged for moving one arm of the human user, wherein the exoskeleton actuator device is arranged to be controlled in accordance with the intended motion and an intended force output from the human intention detection device with the first force sensing device mounted on the opposite side limb. In other embodiments, use for controlling an actuator in a virtual reality and/or gaming setup. In still other embodiments, use for treatment or therapy purposes, e.g. for rehabilitation.

In a sixth aspect, the invention provides a system comprising an HID device according to the first aspect, and an actuator device arranged for being controlled in response to the output from the human intention detection device. Especially, the actuator device, e.g. exoskeleton actuator device, may comprise an elbow joint arranged for being controlled in response to the output from the human intention detection device. Especially, the actuator device, e.g. exoskeleton actuator device, may comprise a shoulder joint arranged for being controlled in response to the output from the human intention detection device. A specific embodiment of said shoulder joint comprises a spherical joint mechanism comprising two revolute joints joined by a double parallelogram linkage. More specifically, the double parallelogram linkage comprises a first linkage part hingedly connected to a first revolute joint at a distal end of the first linkage part and a second linkage part hingedly connected to a second revolute joint at a distal end of the second linkage part,
the first linkage part comprises a first link arm and a second link arm, which first and second link arms are arranged to move parallel to each other,
the second linkage part comprises a third link arm and a fourth link arm, which third and fourth link arms are arranged to move parallel to each other, and
a proximate end of the first linkage part and a proximate end of the second linkage part are mutually hingedly connected.

The system may comprise a robotic arm with at least one actuator arranged to be controlled in response to the output from the human intention detection device, such as a robotic arm arranged for being positioned away from the human user.

The system may comprise an exoskeleton actuator device with at least one actuator arranged to be controlled in response to the output from the human intention detection device, wherein the exoskeleton actuator device is arranged to be worn by the human user.

In a seventh aspect, the invention provides a method of treatment comprising
providing an HID device according to the first aspect,
providing an exoskeleton actuator device arranged to assist in moving one limb of the human user in response to an intended motion and force output from the human intention detection device, such as the exoskeleton actuator device comprising at least one elbow joint actuactor arranged for moving one arm of the human user,
mounting the first force sensing device mounted on the opposite side limb of the limb arranged to be assisted by the exoskeleton actuator device,
controlling the exoskeleton actuator device in accordance with the intended motion and an intended force output from the human intention detection device with, and
performing a training session by the human user moving said opposite side limb in a repetitive manner to cause said one limb to perform a similar movement as said opposite limb.

Such method of training an impaired or otherwise not fully functional limb by control from the corresponding opposite side limb is a useful treatment for various health conditions or diseases. E.g. the method may be used for rehabilitation after a stroke, where it is important to move a not functioning limb quickly after the stroke, so as to train reestablishment of brain function for control of the limb.

It is appreciated that the same advantages and embodiments described for the first aspect apply as well for the further aspects. Further, it is appreciated that the described embodiments can be intermixed in any way between all the mentioned aspects.

### BRIEF DESCRIPTION OF THE FIGURES

The invention will now be described in more detail with regard to the accompanying Figures of which

FIG. 1 illustrates a sensor band,

FIG. 2 illustrates a side view of a strap,

FIG. 3 illustrates placement of sensor band on an arm,

FIG. 4 illustrates an electric circuit diagram,

FIGS. 5a and 5b illustrate graphs showing motion and force patterns,

FIG. 6 illustrates an upper body exoskeleton,

FIG. 7 illustrates a diagram of control structure of an exoskeleton,

FIG. 8 illustrates a flow chart of an algorithm,

FIG. 9 illustrates a HID system for controlling an exoskeleton assistive device,

FIGS. 10a and 10b illustrate photos of a robotic elbow actuator in response to a HID system with upper arm sensors only,

FIGS. 11a-11c illustrate skethes and photos of a shoulder joint and the shoulder joint forming part of an upper body exoskeleton and

FIG. 12 illustrates to the left a sketch of upper arm muscles and indication of preferred force sensing positions, and to the right a photo of a specific prototype with 6 force sensors mounted on a strap or sleave to be mounted on the upper arm.

The Figures illustrate specific ways of implementing the present invention and are not to be construed as being limiting to other possible embodiments falling within the scope of the attached claim set.

### DETAILED DESCRIPTION OF EMBODIMENTS

The proposed HID system to detect the human intention is comprised of the following modules:
1) Sensor Bands
2) Electronics
3) Machine learning algorithm

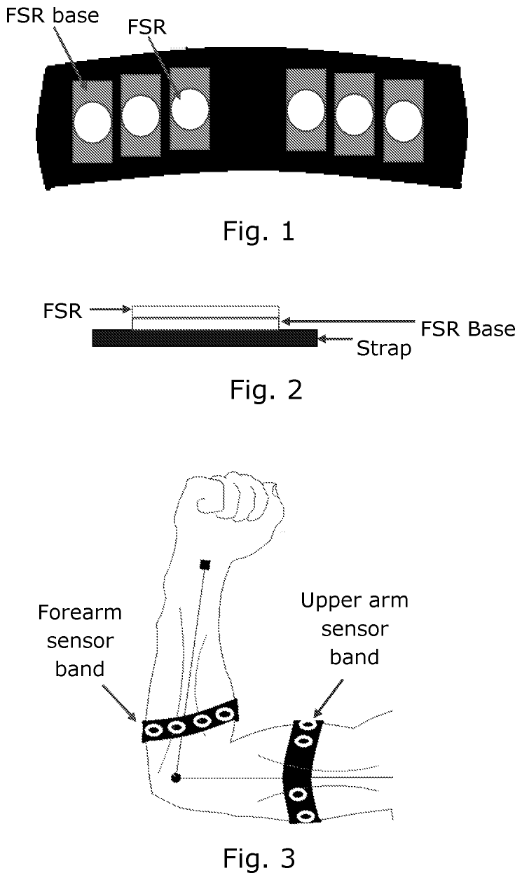

1) Sensor bands are used to read the muscle activity during different movement and muscle force or effort. Each is comprised of a flexible strap with an array of N (5≤N≤10) force sensing resistors (FSRs, e.g. Interlink 402), as illustrated in FIGS. 1 and 2, embedded inside them. FSRs are capable of measuring the force change due to muscle contraction and relaxation. They have been placed in such an order, so that the activity of different muscles groups can be detected. The choice of flexible strap is made, to ensure the comfortability and fixed position on the arm. A small base has been placed between the strap and the FSR to make sure that full surface of FSR gets in contact with the skin.

The sensor bands can be mounted on arm in different ways:
a) One on upper arm and one on forearm (FIG. 3) to detect the motion types, i.e. flexion/extension and pronation/supination, and muscle force or effort.
b) One sensor band only on the upper arm in order to detect the flexion/extension at elbow joint.

The size of the sensor band is adjustable for different users. Moreover, sensor bands can also be placed either on skin or on clothes.

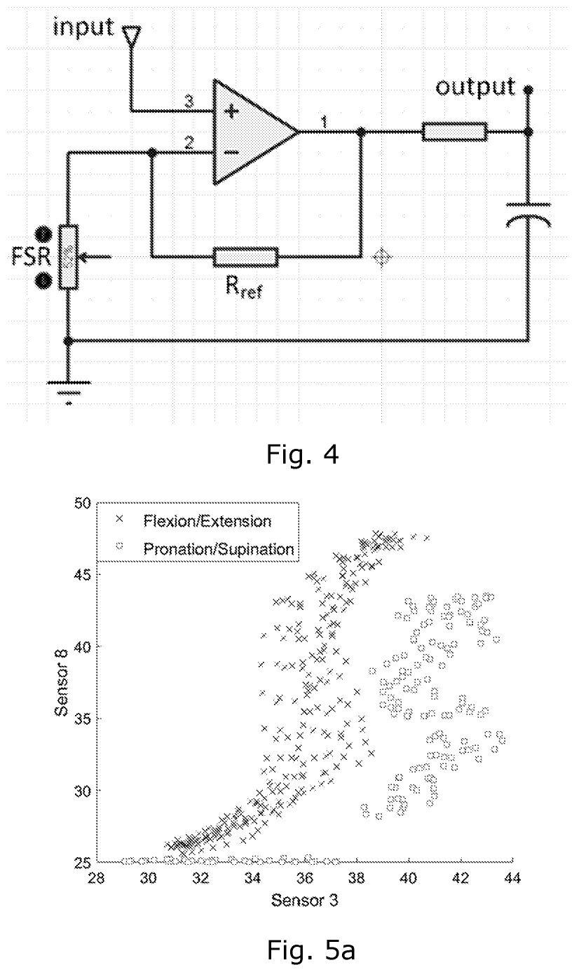

2) Electronics is mainly comprised of non-inverting amplifier (eq. 1) and a low pass filter (FIG. 4).

$${V}_{out} = ( \frac{{R}_{ref}}{FSR} + 1 ) * {V}_{in} \qquad ( 1 )$$

Vᵢₙ(Input) and R_{ref} both sets the sensing range of the FSR. There is a tradeoff between both values. If Vᵢₙ is set to a high value then R_{ref} is set to low and vice versa in order to make use of the maximum range of the FSR. Vᵢₙ is set to a value of 1.2V for each amplifier and R_{ref} is set to a different value for each sensor. This gives a unique advantage of detecting the muscle force with ease. The sensors with high R_{ref} provide a clear distinction between low and medium level of muscle force, while, sensors with low value of R_{ref} are able to distinguish between medium and high muscle force. The low pass filter is designed at the cut off frequency of 150 Hz in order to eliminate the high frequency noise.

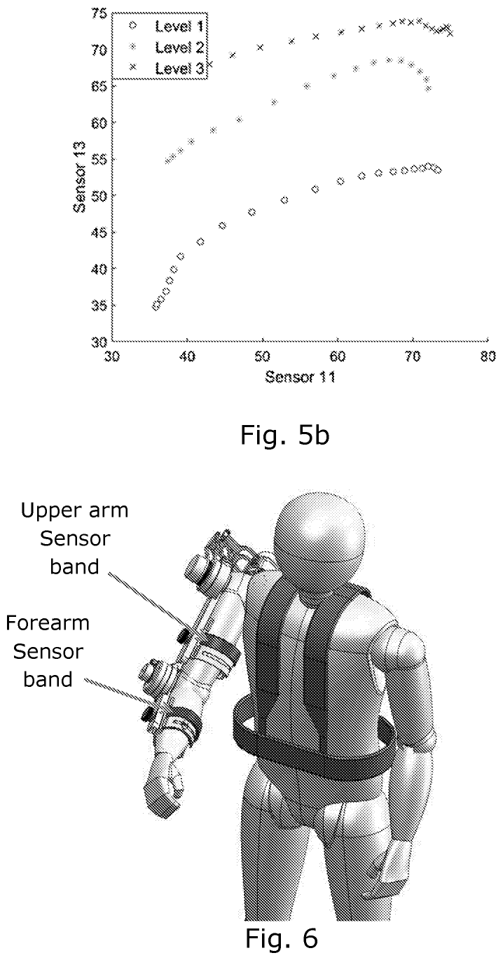

3) A machine learning algorithm is used to intelligently distinguish the patterns register by sensor bands for motion type and muscle force (FIGS. 5a and 5b). The algorithm is implemented on MATLAB, which is running on a microprocessor device. It can also be implemented on C++ or Java.

The flow chart of the algorithm is shown in FIG. 8.

### Control of Exoskeleton Through HID

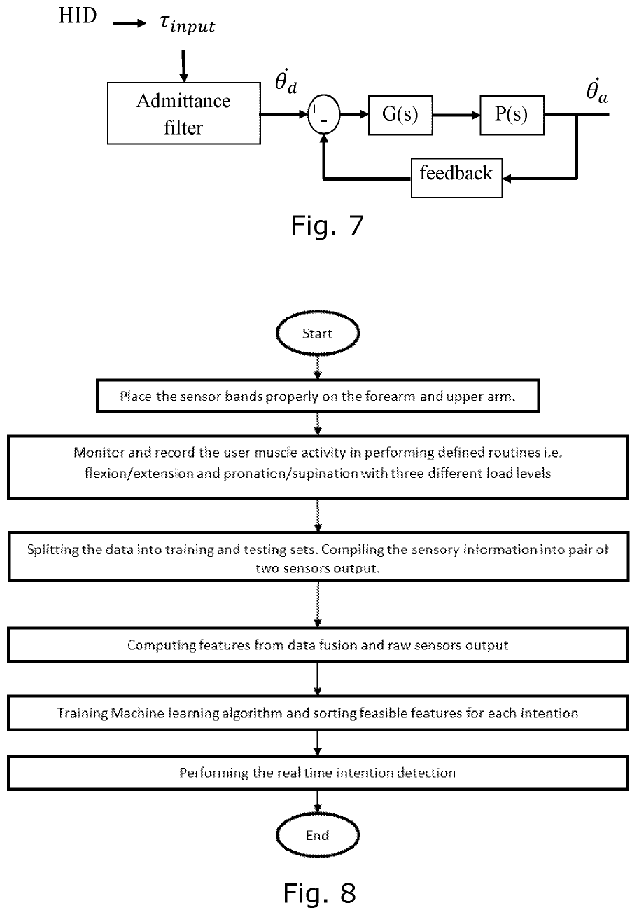

The invention is developed for an upper body exoskeleton (FIG. 6) whose purpose is to assist the human in performing daily activities. The assistance can only be provided if the exoskeleton knows the human intention i.e. how much support a user needs to do any task, which kind of motion the user is doing. By equipping the exoskeleton control with HID (FIG. 7) will enable it to provide the needed physical assistance for any task. As shown in the control algorithm in FIG. 7, HID provides reference torque, namely τᵢₙₚᵤₜ, which is used to generate the reference velocity (\dot{θ}_{d}) as an input to the feedback loop of the velocity control. Compared with previously reported work with EEG and EMG, which is very inconvenient if the human is plugging on the sensors every single time on various places on the skin and then putting up the suit on, the proposed method offers advantages in using. It not only provides better results but also provides an easy interface like a wrist watch. Moreover, a slight misplacement of sensor band also does not affect the results.

### Possible Applications of HID Sensor

### 1. Bilateral Rehabilitation:

The idea is to control the motion of impaired arm by wearing the sensors on the healthy arm. This approach can serve the following two purposes:
The user uses it to accomplish the daily routine tasks that need coordinated motion of both arms e.g. lifting/pulling an object etc.
The user uses it for therapy exercises, which is aimed to bring the movement of impaired arm alive again up to some level.
   2. Virtual reality:

HID can be used to control the device at remote location or in the virtual environment. In this way, the user wearing HID sensors as the controller, with his/her motion reproduced on the remote device or virtual device.

### 3. Control of Shoulder Joint:

The HID can directly detect and control the assistive motion at elbow joint. In certain scenarios the sensor information may be used to support the shoulder complex motion as well.

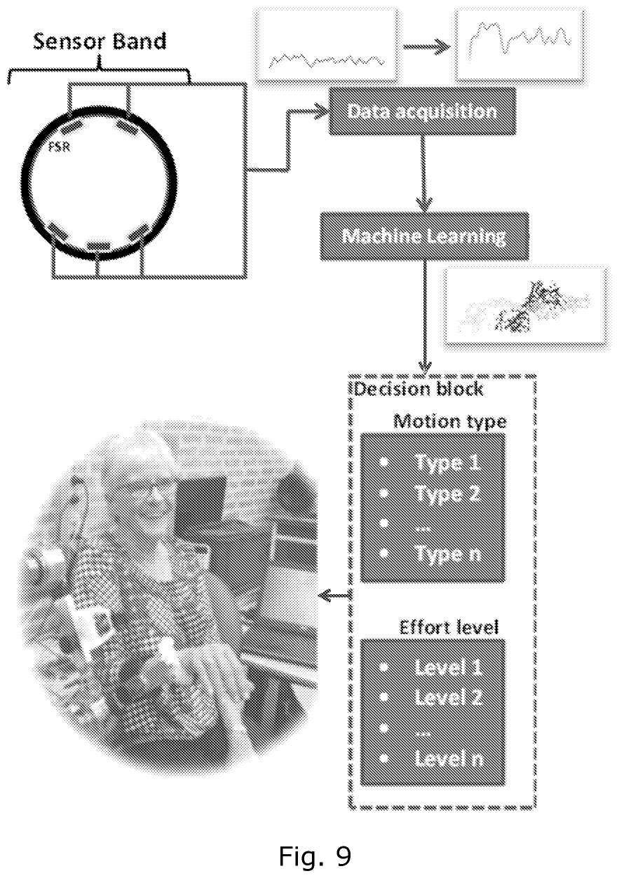

FIG. 9 illustrates the various parts of an HID embodiment. A sensor band with 5 FSRs distributed is to be worn on the upper arm to detect motion and force intention of a user. The FSRs are connected to a data acquisition unit which provides outputs to a machine learning algorithm which finally selects between n types of motions and n levels of force, and these detected motion and force are then used for controlling an assistive exoskeleton actuator device comprising an elbow joint actuator (photo).

In the shown example in FIG. 9, the human user wears the exoskeleton on the same arm as where the sensor band is mounted. However as mentioned, the sensor band may be worn on the opposite arm of where the exoskeleton is mounted, e.g. for treatment purposes, e.g. rehabilitation or training purposes.

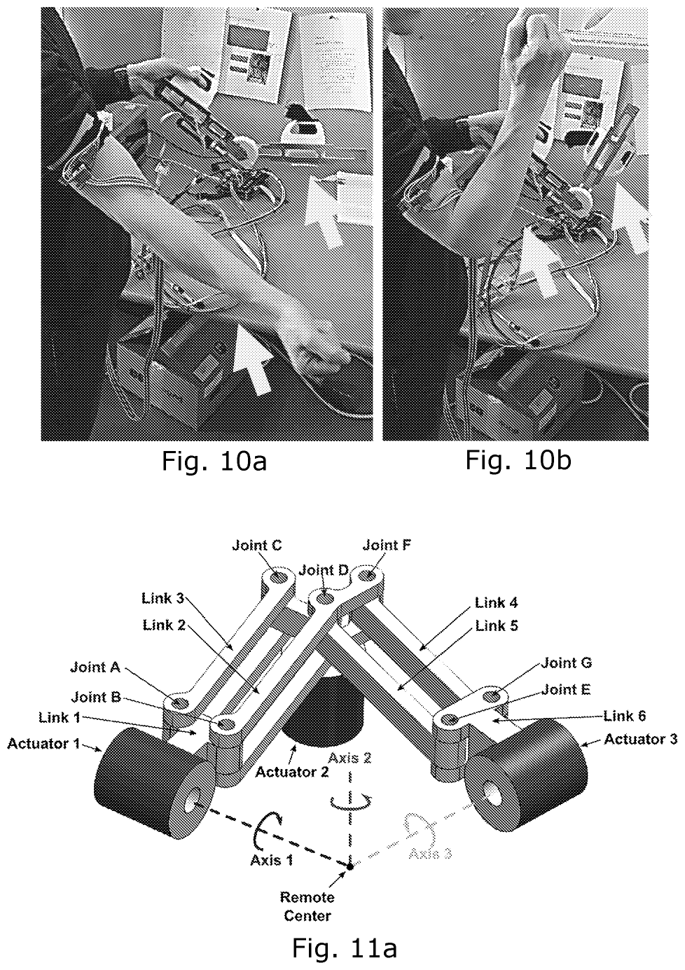

FIGS. 10a and 10b show two photos of a person with a sensor band with FSRs in his upper arm, and a robotic arm with an elbow actuator in a test setup. As seen, the HID system according to the invention successfully detects the intended motion by the person and controls the elbow actuator of the robotic arm accordingly.

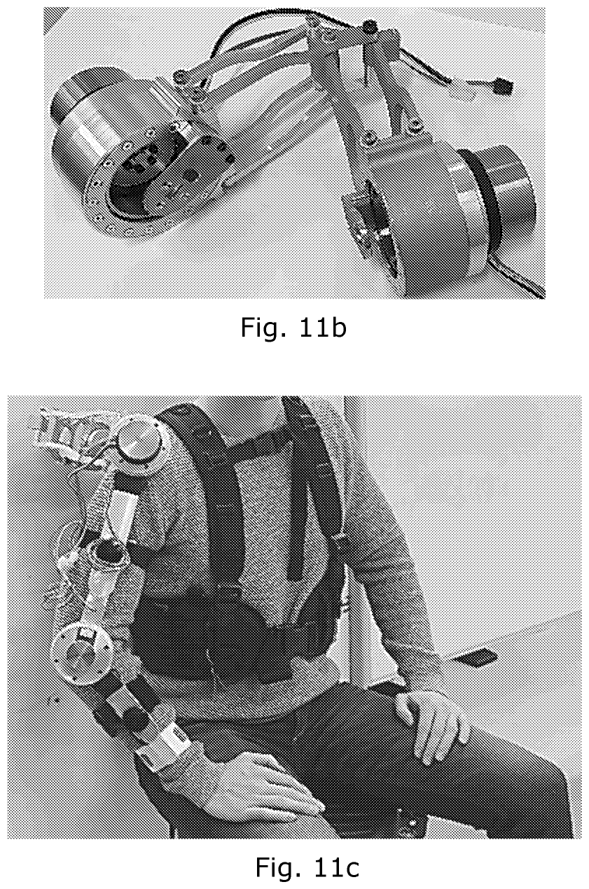

FIGS. 11a and 11b show an example of a controllable shoulder joint which can be controlled with the HID according to the invention. The shoulder joint may form part of an upper body controllable exoskeleton actuator device as shown on the photo in FIG. 11c.

The shown shoulder joint comprises a spherical joint mechanism comprising two revolute joints joined by a double parallelogram linkage, wherein the double parallelogram linkage comprises a first linkage part hingedly connected to a first revolute joint at a distal end of the first linkage part and a second linkage part hingedly connected to a second revolute joint at a distal end of the second linkage part,
the first linkage part comprises a first link arm and a second link arm, which first and second link arms are arranged to move parallel to each other,
the second linkage part comprises a third link arm and a fourth link arm, which third and fourth link arms are arranged to move parallel to each other, and
a proximate end of the first linkage part and a proximate end of the second linkage part are mutually hingedly connected.

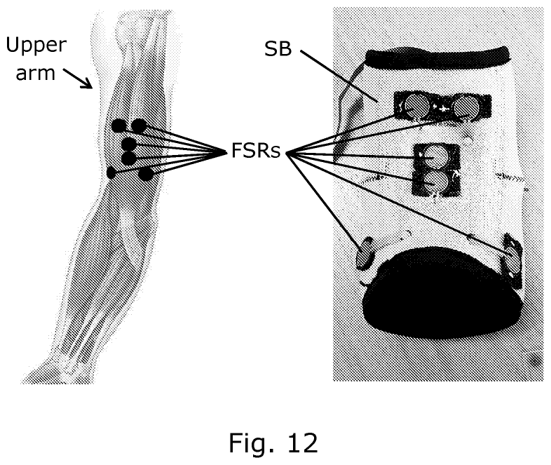

FIG. 12 shows to the right a photo of an embodiment of a force sensing device with 6 FSRs mounted at spatially distributed position on a strap or sleave arranged to be mounted around the upper arm of a human user so as to be positioned along the biceps brachii and brachialis. To the left of FIG. 12 a sketch of the preferred positions of the 6 FSRs in relation to the muscles is shown, i.e. the positions relative to the biceps brachii and the brachialis, when the strap or sleave is mounted as intended on the upper arm. In this embodiment, with the shown 6 different FSR positions, the sensors are placed to allow capturing the 4 motions flexion, extension, pronation and supination using only the shown sensors on the upper arm. Thus, tests have shown that with sensors positioned as shown on the upper arm, sensors positioned on the forearm can be eliminated and still allow the 4 motions: flexion, extension, pronation and supination to be detected.

As seen, the preferred positions of the FSRs are at three different groups in a length direction of the biceps:
two FSRs on an upper portion of the biceps brachii, one on each side, spaced apart by 2-10 cm,
two FSRs on a lower portion of the biceps brachii, one on each side and spaced apart by 6-14 cm, and
two FSRs at different positions in the middle of the biceps brachii, positions at different directions length directions between the two upper and the two lower FSRs.

It is to be understood that more than 6 FSRs can be used to cover yet more positions, if preferred. However, the 6 described positions have proven sufficient to provide reliable detection of all of flexion, extension, pronation and supination motions.

The muscles of the forearm perform many different types of motions, i.e. open and close of fist, wrist flexion/extension and many others, besides pronation/supination. If only pronation/supination and elbow's flexion/extension is of interest, then this can be achieved alone by the described upper arm sensor device. The muscles at the upper arm are majorly involved in elbow flexion/extension and forearm pronation/supination. They are not involved in the motion performed at wrist joint. Hence, there will be less disturbance on upper arm muscles by the wrist motions, and the results can be better if there is focus on upper arm muscles only.

However, it is to be understood that this upper arm embodiment could also be used in combination with a forearm force sensing device with one or more FSRs, if additional motions should be detected. I.e. if detection of motions at wrist joint is of interest, it can be done by a forearm sensing device with one or more FSRs in combination with the above described upper arm sensing device.

The FSRs may specifically be such as the Interlink 402, however it is to be understood that other FSRs may be used as well. Further, other types of force sensors may be used than FSRs.

The strap or sleave may be arranged to tighten around the upper arm to provide a proper fit, and/or it may be formed by an elastic material, e.g. an elastic garment that will ensure a proper fit. Especially, the strap or sleave may be made of an elastic material, e.g. an elastic garment, of a predetermined circumference and arranged for mounting by the human user pulling the strap or sleave up to the forearm and turning it to provide proper positions of the FSRs.

The detection algorithm design to be used with the above described upper arm sensor embodiment is similar to what has already been described.

In the following, preferred methods for control of shoulder and elbow assistance level for exoskeletons will be described. Effort level estimated for elbow joint can be used to estimate the assistance required at the shoulder joint.

Effort Level estimation for elbow joint can be estimated by considering both the muscle contraction forces (MCF), measured by FSRs embedded in an upper arm sensor device, and by measuring elbow joint angle. It is known that muscle contraction and stiffness is directly proportional to weight of an object in hand. The algorithm utilizes the foretold information in the following way to compute the effort level. The first part is the training session, which comprises the following steps:
1) Two regression models (RM⁵ and RM⁰ are developed, that relates the MCF to elbow joint angle.
2) First regression model RM⁵ maps the MCF to complete range of motion of elbow joint for a 5 kg load.
3) The second one RM⁰ relates the same parameters without having any load.

In the testing part, the algorithm first estimates that for a given joint angle what would be the MCFs (F⁵, F⁰ ) for the case of e.g. a 5 kg and a 0 kg load using the regression models developed in the training session. In the next step, the distance relation is utilized to map the actual value of MCF (F^{a}), measured at the current stage, in between F⁵and F⁰ in order to estimate the effort level. The equation to measure the effort level is:

EL=(1−(F⁵(θ)−F^{a})/(F⁵(θ)−F⁰(θ)))*E_{range}

Here F⁵(θ) and F⁰(θ) represent the forces F⁵ and F⁰ as a function of elbow joint angle related through regression models RM⁵ and RM⁰ respectively. E_{range} represents the range of effort level, which in the specific case is 5.

Assistance at shoulder joint may be computed by updating the gravity compensation torque model for shoulder joint, which is given by:

τ=g(mₑ, θₑ, mₛ, θₛ)

Here mₑ and θₑ represent the mass and joint angle of elbow joint, and mₛ, θₛ represent the mass and joint angle of shoulder joint, respectively.

As, it is clear that EL estimation model maps 0 kg load to EL=0, and 5 kg load to EL=5, so EL is basically representing the amount of load the human is carrying. Hence, the estimated EL is used to update the mass parameters, mₑ, of elbow joint of the exoskeleton, which ultimately updates the gravity torque for the shoulder joint for the given joint angles for both elbow and shoulder. This is how assistance may be provided to the shoulder point.

To sum up: the invention provides a novel device and method for human intention detection (HID). In preferred embodiments, it makes use of an array of force sensing resistors (FSRs) which are embedded inside a flexible band, which is capable of reading the muscle activity for different motion type and muscle force in a human user. In one implementation of the invention two of such bands are attached to the forearm and the upper arm. From the readings of the sensors, the patterns for motion type and muscle force are then distinguished autonomously by machine learning, e.g. a Support Vector Machine (SVM) algorithm, neural networks (NN). The method is advantageous e.g. the detection of dexterous motion of the arms, upon which assistive exoskeleton can be controlled for motion assistance. The invention can also be applicable to hand gestures recognition and bilateral rehabilitation, besides this the invention can be used to control lower body exoskeleton as well.

Although the present invention has been described in connection with the specified embodiments, it should not be construed as being in any way limited to the presented examples. The scope of the present invention is to be interpreted in the light of the accompanying claim set. In the context of the claims, the terms “including” or “includes” do not exclude other possible elements or steps. Also, the mentioning of references such as “a” or “an” etc. should not be construed as excluding a plurality. The use of reference signs in the claims with respect to elements indicated in the FIGS. shall also not be construed as limiting the scope of the invention. Furthermore, individual features mentioned in different claims, may possibly be advantageously combined, and the mentioning of these features in different claims does not exclude that a combination of features is not possible and advantageous.

1. A human intention detection device configured to detect an intended motion of a human user, and to generate an output the method comprising:
a first force sensing device configured to mount around an upper part of a limb part of the human user so as to allow sensing of muscle contraction, wherein the first force sensing device comprises a plurality of force sensors spatially distributed to allow detection of different muscle parts of the human user's upper limb part and to generate outputs accordingly, and
a processor device configured to receive said outputs from the force sensors of the first force sensing device, wherein the processor device comprises a processor configured to execute a detection algorithm in response to said outputs from the force sensors of the first force sensing device, and wherein the processor device is configured to output in real-time an intended motion and an intended force according to an output from the detection algorithm.

2-39. (canceled)

40. The human intention detection device according to claim 1, comprising a second force sensing device configured to mount around a lower part of the same limb as said first force sensing device, wherein the second force sensing device comprises a plurality of force sensors spatially distributed to allow detection of different muscle parts of the human user's lower part of the limb, and to generate outputs accordingly and wherein the processor device is configured to receive outputs from the force sensors of the second force sensing device and to output in real-time the intended motion and the intended force in response to outputs from the force sensors of the first and second force sensing devices.

41. The human intention detection device according to claim 1, wherein the first force sensing device is configured to mount on the upper arm of the human user.

42. The human intention detection device according to claim 40, wherein the first force sensing device is configured to mount on the upper arm of the human user and the second force sensing device is configured to mount on the forearm of the human user.

43. The human intention detection device according to claim 1, configured to discriminate between a plurality of levels of said muscle contraction activity.

44. The human intention detection device according to claim 1, wherein the force sensing device comprises a strap with the plurality of force sensors arranged on a line, on the side of the strap, which is configured to face the human user's upper limb part.

45. The human intention detection device according to claim 1, wherein the plurality of force sensors are Force Sensitive Resistor type sensors.

46. The human intention detection device according to claim 1, wherein the detection algorithm implements a support machine vector (SVM) or a neural network (NN) for classifying the intended motion and the intended force.

47. The human intention detection device according to claim 46, wherein the intended motion and the intended force are classified by computing required features through data fusion and raw sensor values.

48. The human intention detection device according to claim 1, wherein the detection algorithm comprises a training session, wherein the human user performs a plurality of intended motions for generating test data, wherein the detection algorithm further comprises computing accuracy in response to the test data, and wherein the detection algorithm also comprises selecting features, which can be obtained from both data fusion and original signals from FSRs for use in outputting said real-time intended motion and intended force based on the training sessions results.

49. The human intention detection device according to claim 1, wherein information obtained from force reading and data fusion, is used to detect the intended motion and the intended force.

50. The human intention detection device according to claim 1, wherein the first device comprises a strap or a sleeve configured to mount around an upper arm of the human user, wherein a plurality of force sensing devices are spatially distributed on the strap or sleeve so as to allow detection of contraction at a plurality of positions along biceps brachii and/or brachialis when the strap or sleeve is mounted on the upper arm of the human user.

51. The human intention detection device according to claim 1, comprising 5-8 FSRs spatially distributed to cover at least three different positions along a length of the biceps brachii, as well as, at least two different positions perpendicular to the length of the biceps brachii.

52. The human intention detection device according to claim 51, wherein the detection algorithm is configured to output one or more intended motions selected from: flexion, extension, pronation or supination, in response to outputs from the plurality of force sensing devices.

53. The human intention detection device according to claim 1, wherein the first force sensing device comprises a strap or sleeve on which the plurality of force sensors are mounted at different positions.

54. A method for detecting an intended human motion, the method comprising:
sensing a muscle contraction activity with a plurality of force sensors arranged on an upper part of a limb, wherein said sensors are spatially distributed to allow detection of different muscle parts of the human user's upper part of said limb,
executing a detection algorithm on a processor in response to outputs from the force sensors, and
outputting in real-time an intended motion and an intended force according to an output from the detection algorithm.

55. A computer executable program code arranged to perform the method according to claim 54, when executed on a processor.

56. A method of using the device of claim 1 for controlling a robotic arm comprising an actuator, which is configured to be worn by a human user comprising providing the human intention detection device of claim 1 to a human user, wherein the human intention detection device is integrated to control said robotic arm comprising an actuator, which is configured to be worn by the human user.

57. The method according to claim 56, wherein said robotic arm comprising an actuator, which is configured to be worn by the human user is further configured for controlling an actuator in a virtual reality and/or gaming setup.

58. A system comprising a human intention detection device according to claim 1, and an actuator device configured to control said actuator device in response to the output from the human intention detection device.
